(19) United States
(12) Patent Application Publication
(10) Pub. No.: US 2019/0315816 A1
(43) Pub. Date: Oct. 17, 2019

(54) PROTRANSDUCINE-C: GENE TRANSFER ACTIVATOR

(71) Applicant: PHARIS BIOTEC GMBH, Hannover (DE)

(72) Inventors: Wolf-Georg FORSSMANN, Hannover (DE); Rudolf RICHTER, Hannover (DE)

(21) Appl. No.: 16/347,762

(22) PCT Filed: Nov. 8, 2017
(86) PCT No.: PCT/EP2017/078600

§ 371 Date: May 6, 2019

(30) Foreign Application Priority Data
Nov. 9, 2016 (EP) 16197999.2
Nov. 10, 2016 (EP) 16198256.6

(51) Int. Cl. C07K 14/435; C12N 7/00; C12N 15/86

(52) U.S. Cl. CPC C07K 14/435; A61K 38/00; C12N 15/86; C12N 7/00

(57) ABSTRACT

Polypeptide having the sequence

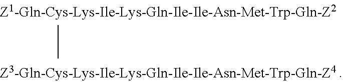

The present application relates to a polypeptide, an N-terminally protected polypeptide, a medicament containing said polypeptide, said polypeptide for use in gene therapy, a process for enhancing the infection of a cell by a genetically engineered virus construct, the use of said polypeptide for amplification for transfection or transduction.

In recent years, the importance of genetic engineering has increased because of enormous advances in applied methods, since not only the production of protein-peptide drugs, but also the transfection of cells with stable genes as a laboratory tool and finally the incorporation of genes into cells as a surrogate for gene defects is foreseeable to be of great importance to the therapy of numerous diseases.

The incorporation of gene material for changing specific cell functions has become an indispensable tool of biological-medical basic and applied research since the cloning of the first human genes and recombinant production. There is a constant progress in the methods for gene incorporation that leads to an optimization of gene transfer. These are experiences gathered over long years of a history that proceeded very slowly at first.

Already before the elucidation of the function of deoxyribonucleic acid (DNA) in 1953 by F. Crick and J. Watson, F. Griffith had succeeded in the end of the 1920's in experiments in transforming non-pathogenic pneumococcus strains into pathogens. This transformation was due to a lucky circumstance, because the pneumococci possessed a rare natural competence of DNA uptake. A selected incorporation of DNA into prokaryotes, a so-called transduction, was successfully performed using phages by, among others, J. Lederberg, M. Delbrück and S. Luria. With the establishment of cell culturing, i.e., the culturing of eukaryotic cells under in vitro conditions, a number of physical and chemical methods for transfection were developed. The physical methods, which are more frequently employed, but require costly equipment, include electroporation and microinjection; these competed with the chemical methods, which are more simply to apply, such as the calcium phosphate precipitation method, which has been in use since the 1980's, or the methods based on cationic lipids or cationic polymers, which were widespread in the early 1990's. However, the use of such methods was always dependent on the cells or the DNA. Also, the DNA incorporated into the cells was generally extrachromosomal (transient transfection) and thus was not passed on to the daughter cells. However, it was known that phages (e.g., lambda phage) are also capable of integrating their DNA into the host genome (prophage, lysogenic infection pathway). From here, it was only a small step to the (1981/1982) “Establishment of Retroviruses as Gene Vectors” (by Doehmer et al. and Tabin et al.). Viruses are species-specific and organ/tissue-specific, which is why not all viruses infect all (eukaryotic) cells. Alterations of the viral envelope (replacement of glycoproteins, so-called pseudo-typed viruses) as well as additions of, mostly cationic, peptides are intended to enhance transduction efficiency. The first enhancers of the uptake of viral particles attracted attention in the study of HIV. During analyses of in vitro infection by means of a specific cell test, the inhibition of the fusion of HIV by blood filtrate peptides was observed (Munch et al., VIRIP).

It has been shown that these fragments of proteins, which are surprisingly naturally occurring, form fibrous structures in human sperm as an enhancer or “Semen Derived Enhancer of Virus Infection” (SEVI) that are characterized as amyloid fibrils. Such nanofibrils enhance the attaching of viruses to their target cells and increase the rate of viral infection by several powers of ten.

This fact was utilized for improving a retroviral gene transfer for basic research and possible future therapeutic applications. Thus, it could be shown that lentiviral and gamma-retroviral vectors, which are used for gene therapy, exhibit a multiply increased gene transfer rate in the presence of the SEVI protein in different cell types, such as human T cells, cervical carcinoma cells, leukemia cells, hematopoietic stem cells, and embryonic stem cells (Wurm et al., J Gene Med. 2010, 12, 137-46; Wurm et al., Biol Chem. 2011, 392, 887-95).

Studies for the development of further enhancers, such as SEVI and seminogeline, lead to the assumption that peptides from viral envelope proteins could also be suitable as enhancers of transfection, which surprisingly had an unexpectedly high success (Maral Yolamanova et al., Nature Nanotechnology, Vol. 8, No. 2, pp. 130-136). Thus, for example, it could be shown that HIV viruses that were preincubated with different concentrations (1-100 μg/ml) of protransducine-A (synonym: EF-C) exhibit an infection rate in reporter cells that is higher by several powers of ten in reporter cells. As the mechanism of action, it was assumed that EF-C forms fibrillar structures that are capable of binding, concentrating viruses and accordingly to multiply the entry of the viruses into a cell. In addition to the infection with viral particles, EF-C enhances, with high efficiency, the transduction of lentiviral and retroviral particles into a wide variety of human cell types (T cells, glia cells, fibroblasts, hematopoietic stem cells), which are applied in gene therapy (Maral Yolamanova et al., Nature Nanotechnology, Vol. 8, No. 2, pp. 130-136). EP 2 452 947 A1 also relates to protransducine-A.

Because of the increasing importance of genetic engineering as set forth above, more effective enhancers of gene transfer are desirable. The problem underlying the invention is to provide an improved enhancer of gene transfer.

Surprisingly, it has been found that the problem underlying the invention is solved by polypeptides having a sequence similarity of at least 80% or 90%, especially 95%, with the sequence

Z¹-Gln-Cys-Lys-Ile-Lys-Gln-Ile-Ile-Asn-Met-Trp-
Gln-Z²
Z³-Gln-Cys-Lys-Ile-Lys-Gln-Ile-Ile-Asn-Met-Trp-
Gln-Z⁴,

wherein

Z¹ or Z³ independently represent the N-terminal end of the polypeptide, or independently are the amino acids Leu or Ser or the following peptides

### Ser-Asn, Ser-Asn-Asn, Ser-Asn-Asn-Ile, Ser-Asn-Asn-Ile-Thr, Thr-Leu, Ile-Thr-Leu, Asn-Ile-Thr-Leu, Asn-Asn-Ile-Thr-Leu, or Ser-Asn-Asn-Ile-Thr-Leu,

Z² or Z⁴ independently represent the C-terminal end of the polypeptide, or independently are the amino acids Gly or Glu or the following peptides

Glu-Val,
Glu-Val-Gly,
Glu-Val-Gly-Lys,
Glu-Val-Gly-Lys-Ala,
Glu-Val-Gly-Lys-Ala-Met,
Glu-Val-Gly-Lys-Ala-Met-Tyr,
Glu-Val-Gly-Lys-Ala-Met-Tyr-Ala,
Glu-Val-Gly-Lys-Ala-Met-Tyr-Ala-Pro,
Glu-Val-Gly-Lys-Ala-Met-Tyr-Ala-Pro-Pro,
Glu-Val-Gly-Lys-Ala-Met-Tyr-Ala-Pro-Pro-Ile,
Glu-Val-Gly-Lys-Ala-Met-Tyr-Ala-Pro-Pro-Ile-Glu,
Glu-Val-Gly-Lys-Ala-Met-Tyr-Ala-Pro-Pro-Ile-Glu-
Gly,
Glu-Gly,
Ile-Glu-Gly,
Pro-Ile-Glu-Gly,
Pro-Pro-Ile-Glu-Gly,
Ala-Pro-Pro-Ile-Glu-Gly,
Tyr-Ala-Pro-Pro-Ile-Glu-Gly,
Met-Tyr-Ala-Pro-Pro-Ile-Glu-Gly,
Ala-Met-Tyr-Ala-Pro-Pro-Ile-Glu-Gly,
Lys-Ala-Met-Tyr-Ala-Pro-Pro-Ile-Glu-Gly,
Gly-Lys-Ala-Met-Tyr-Ala-Pro-Pro-Ile-Glu-Gly,
Val-Gly-Lys-Ala-Met-Tyr-Ala-Pro-Pro-Ile-Glu-Gly,
or
Glu-Val-Gly-Lys-Ala-Met-Tyr-Ala-Pro-Pro-Ile-Glu-
Gly.

The term “sequence similarity” herein means the similarity between proteins (amino acid sequence homology) because of identical component sequences in more or less extended partial sections of the protein. The sequence similarity is expressed in percent identical positions when two peptide chains are compared, wherein 100% sequence similarity means a complete identity of the chain molecules compared. In proteins, 5% (100/20) identical positions correspond to the statistical expectation and thus cannot be used for deriving relationships.

The improvement that can be achieved by the polypeptide according to the invention is associated with an increased efficiency of gene transduction in cells, which could be used for therapeutic purposes. For example, a more efficient gene transduction in cells for therapeutic use can reduce the amount of viral particles used for gene transduction. Further, the number of infection cycles necessary for an efficient transduction can be reduced. The polypeptide according to the invention as a transduction enhancer can reduce the duration of in vitro culturing for multiplying the gene-modified cells, the amount of cells to be collected from the patient (e.g., by leukapheresis), and in some cases enable an efficient and non-toxic in vivo gene transduction by reducing the viral load in vivo. Further, the quick handling of an efficient transduction enhancer reduces the load on the cells to be transduced.

The polypeptide according to the invention can be formed from homologous or heterologous monomers. In particular, the polypeptide according to the invention is a dimer consisting of two identical monomers bonded together through a disulfide bridge. In one embodiment of the invention, the polypeptide according to the invention has the following structure:

Z¹-Gln-Cys-Lys-Ile-Lys-Gln-Ile-Ile-Asn-Met-Trp-
Gln-Z²
|
Z¹-Gln-Cys-Lys-Ile-Lys-Gln-Ile-Ile-Asn-Met-Trp-
Gln-Z²,

wherein Z¹ and Z² have the same meanings as defined above.

In particular, the invention also relates to a polypeptide having a sequence similarity of at least 40%, especially 90%, with the sequence

Gln-Cys-Lys-Ile-Lys-Gln-Ile-Ile-Asn-Met-Trp-Gln
|
Gln-Cys-Lys-Ile-Lys-Gln-Ile-Ile-Asn-Met-Trp-Gln.

According to the invention, the designation “polypeptide according to the invention” also means those related polypeptides that are formed by varying the amino acids in the polypeptide chain of the polypeptide according to the invention, but still have a comparable and sufficient effectiveness, which can be determined, for example, in the following bioassay.

The transduction efficiency can be tested using 293 T cells as target cells and using lentiviral and retroviral vectors encoding green fluorescent protein (GFP). Protransducine-C is employed in an assay in a concentration series, for example, at a concentration of from 0.01 to 1000 μg/ml. In particular, the target cells are employed at a concentration of 10³ to 10⁷ cells/ml.

The batch is incubated for 8 to 16 hours, followed by washing the cells. The transduction efficiency is subsequently measured on the basis of the GFP-positive cells using flow cytometry.

A polypeptide having sequence similarity (homologous polypeptide) is, in particular, a polypeptide related with the amino acid sequence of the polypeptide according to the invention in which substitutions or deletions of amino acids were performed in the amino acid chain to the extent as mentioned. In particular, substitutions of amino acids having similar properties, for example, similar polarities, are possible. Thus, substitutions among arginine and lysine; glutamic acid and aspartic acid; glutamine, asparagine and threonine; glycine, alanine and proline; leucine, isoleucine and valine; tyrosine, phenylalanine and tryptophan; as well as serine and threonine are widespread.

“Sequence homology” herein also means the following: The polypeptide according to the invention consists of two monomers having sequence similarity or identical monomers, each containing the amino acid cysteine, through which the two monomers are linked together through a disulfide bridge. In particular, the amino acid cysteine occupies position 2 of the monomer. Alternatively, other covalent bonds between two amino acids may also be used for bridging the monomers.

In position 1 of the sequence, the amino acid glutamine is preferably found. In positions 3 and 5, basic amino acids are preferably found, more preferably lysine. In positions 1, 4, 6, 7, 8, 9, 10, 11 and 12, neutral amino acids are mostly found. The sequence may be extended or truncated N-terminally and/or C-terminally. On the N-terminal side, the sequence of the monomer may be extended by C-terminal portions of or the entire amino acid sequence NH₂-Ser-Asn-Asn-Ile-Thr-Leu-COOH. On the C-terminal side, the sequence of the monomer may be extended by N-terminal portions of or the entire amino acid sequence NH₂-Glu-Val-Gly-Lys-Ala-Met-Tyr-Ala-Pro-Pro-Ile-Glu-Gly-COOH. The peptide dimers have in common that they form insoluble aggregates in aqueous solutions. The monomers consist of 4 to 25 amino acids, preferably of 10 to 20 amino acids.

Polypeptides having sequence similarity exhibit a sequence similarity of at least 40%, preferably 50%, more preferably a sequence similarity of 60% to 70% or 80%, especially 90% or 95%. In structural terms, the dimerization and at least two basic amino acids can be found in the molecules having sequence similarity. Further, the molecules having sequence similarity have in common that they form insoluble aggregates in aqueous solutions and enhance the transduction of target cells with lentiviral or retroviral vectors.

In an alternative embodiment of the invention, at least one N-terminal end of the two amino acid chains forming the polypeptide according to the invention is modified with a chemical group selected from the group consisting of one or two alkyl groups, such as methyl, ethyl, propyl or butyl groups, an acyl group, such as an acetyl or propionyl group. Alternatively, the N-terminal amino acid may be substituted by a pyroglutamic acid, so that pyroglutamic acid

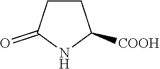

forms the N-terminal end.

The invention also relates to an auxiliary agent for the precipitation of viruses, containing at least one polypeptide according to the invention. By using the polypeptide according to the invention as a precipitation auxiliary, the isolation of viruses by centrifugation can be facilitated significantly, because the centrifugation times to be applied can be significantly shortened.

The invention also relates to a medicament containing at least one polypeptide according to the invention.

The invention also relates to the use of a polypeptide according to the invention in gene therapy for treating genetically caused diseases. In medicine, “gene therapy” means the introduction of genes into cells or tissues of a human in order to treat hereditary diseases or gene defects.

The present invention also relates to a process for enhancing the infection of a cell with a virus, comprising the steps of:
providing the polypeptide according to claim 1 or 2 dissolved in an organic solvent;
adding the polypeptide to an aqueous solution to form insoluble aggregates of the polypeptide;
mixing the solution from the preceding step; and
culturing cells in the presence of at least one polypeptide according to the invention.

The present invention also relates to the use of at least one polypeptide according to the invention for enhancing the infection of cells by a virus.

The present invention also relates to a composition containing at least one polypeptide according to the invention.

The polypeptide according to the invention can be synthesized, for example, by the method according to Merrifield with Fmoc-protected amino acids.

This method is performed with Fmoc-protected derivatives, i.e., with (9-fluorenylmethoxycarbonyl)-protected amino acids in a stepwise solid phase synthesis according to the Merrifield principle, especially on a Wang resin preloaded with Fmoc-L-glutamine (0.59 mmol/g, 100-200 mesh) as a solid support on an ABI-433 synthesizer. The activation of the Fmoc-L-amino acids, which are typically employed in tenfold molar excess, is performed with [(2-(1H-benzotriazol-1-yl)-1,1,3,3-tetramethyluronium hexafluorophosphate] (HBTU, 100 mmol/1) with the addition of 0.5 M 1-hydroxybenzotriazole (HOBt) and 2 M diisopropylethylamine (DIEA) in N-methyl-2-pyrrolidinone (NMP) at room temperature.

The acylation reactions have an individual duration of 45 minutes, and the cleavage of the Fmoc protecting group with 20% piperidine has a duration of 15 minutes.

The following amino acid derivatives and related orthogonally acid-sensitive side chain protective groups are employed for the synthesis:

Fmoc-L-Asn(Trt), Fmoc-L-Cys(Trt), L-pGlu, Fmoc-L-Gln(Trt), Fmoc-L-Ile, Fmoc-L-Lys(Boc), Fmoc-L-Met, and Fmoc-L-Trp(Boc).

After cleavage of the resin support from the peptidyl resin with 94% trifluoroacetic acid (TFA), 3% ethanedithiol (EDT) and 3% demineralized water, the raw peptide is precipitated in cold tert-butyl methyl ether, the raw peptide is centrifuged off as a pellet, and the supernatant is discarded.

The subsequent chromatographic purification of the raw peptide is effected by a preparative method by gradient elution.

The difference of protransducine-A according to EP 2 452 947 A1 and protransducine-B according to WO 2014/177635 A1 as compared to protransducine-C resides in the fact that protransducine-C consists of two peptide monomers according to the invention, which are covalently linked to a peptide dimer through a disulfide bridge.

### EXAMPLE

Transduction of 293 T Cells with Protransducine-C

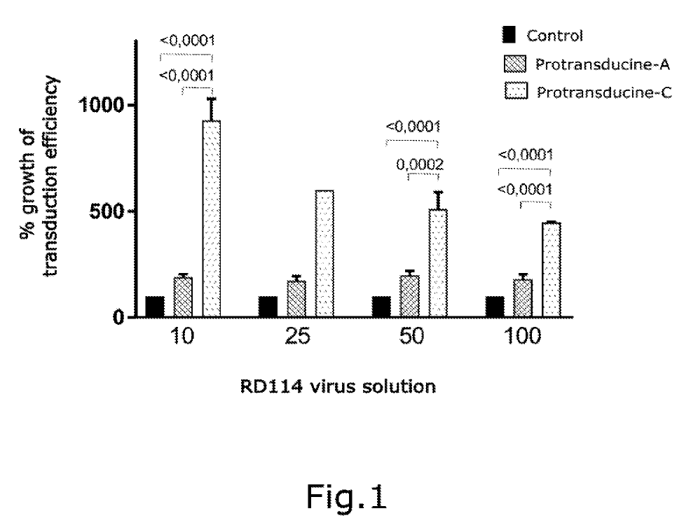

293 T cells are sown in 12-well culturing plates and cultured for 16 hours. Per well, 200,000 293 T cells are employed. After 16 hours, a vial with 1.0 mg of protransducine-C and a vial with 1.0 mg of protransducine-A are admixed each with 100 μl of DMSO, and protransducine-S and protransducine-A are completely dissolved with it. Thereafter, 900 μl of PBS is added in each vial. Fibrils are formed within 3 min Subsequently, protransducine-C or protransducine-A is added at a final concentration of 25 μg/ml to the culture wells. Then different amounts of RD114 Virus Stock (10 μl, 25 μl, 50 μl, 100 μl) are added, and the remaining volume is filled up with DMEM to 500 The RD114 viruses employed encode the Green Fluorescent Protein. In order to enhance the transduction rate, the batches are centrifuged at 600 g at 25° C. for 1 hour. After 6 hours, 500 μl of cell culture medium is added. The cell culture medium is replaced 24 hours after the transduction. The transduction efficiency is determined in flow cytometry 48 hours after the transduction by determining the expression of the Green Fluorescent Protein in the 293 T cells. In the experiments, a significantly higher potency of protransducine-C for enhancing the transduction is seen as compared to protransducine-A and the control (FIG. 1).

Protransducine-B has a similar transduction efficiency as protransducine-A, so that the transduction efficiency of protransducine-C is also significantly increased as compared to protransducine-B, similar to the results according to FIG. 1.

1. A polypeptide having a sequence similarity of at least 80% with the sequence 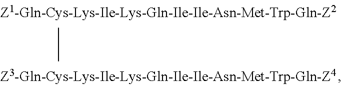
wherein
Z¹ or Z³ have the same meaning or independently represent the N-terminal end of the polypeptide, or independently are the amino acids Leu or Ser or the following peptides
Ser-Asn, Ser-Asn-Asn, Ser-Asn-Asn-Ile, Ser-Asn-Asn-Ile-Thr, Thr-Leu, Ile-Thr-Leu, Asn-Ile-Thr-Leu, Asn-Asn-Ile-Thr-Leu, or Ser-Asn-Asn-Ile-Thr-Leu,
Z² or Z⁴ have the same meaning or independently represent the C-terminal end of the polypeptide, or independently are the amino acids Gly or Glu or the following peptides
Glu-Val, Glu-Val-Gly, Glu-Val-Gly-Lys, Glu-Val-Gly-Lys-Ala, Glu-Val-Gly-Lys-Ala-Met, Glu-Val-Gly-Lys-Ala-Met-Tyr, Glu-Val-Gly-Lys-Ala-Met-Tyr-Ala, Glu-Val-Gly-Lys-Ala-Met-Tyr-Ala-Pro, Glu-Val-Gly-Lys-Ala-Met-Tyr-Ala-Pro-Pro, Glu-Val-Gly-Lys-Ala-Met-Tyr-Ala-Pro-Pro-Ile, Glu-Val-Gly-Lys-Ala-Met-Tyr-Ala-Pro-Pro-Ile-Glu, Glu-Val-Gly-Lys-Ala-Met-Tyr-Ala-Pro-Pro-Ile-Glu-Gly, Glu-Gly, Ile-Glu-Gly, Pro-Re-Glu-Gly, Pro-Pro-Ile-Glu-Gly, Ala-Pro-Pro-Ile-Glu-Gly, Tyr-Ala-Pro-Pro-Ile-Glu-Gly, Met-Tyr-Ala-Pro-Pro-Ile-Glu-Gly, Ala-Met-Tyr-Ala-Pro-Pro-Ile-Glu-Gly, Lys-Ala-Met-Tyr-Ala-Pro-Pro-Ile-Glu-Gly, Gly-Lys-Ala-Met-Tyr-Ala-Pro-Pro-Ile-Glu-Gly, Val-Gly-Lys-Ala-Met-Tyr-Ala-Pro-Pro-Ile-Glu-Gly, or Glu-Val-Gly-Lys-Ala-Met-Tyr-Ala-Pro-Pro-Ile-Glu-Gly.

2. The polypeptide according to claim 1, wherein said dimer is formed from homologous or heterologous monomers.

3. The polypeptide according to claim 1, having the amino acid sequence

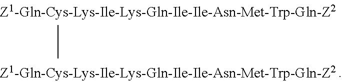

4. The polypeptide according to claim 1, to comprising a sequence of at least 80% similarity with the sequence

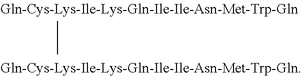

5. The polypeptide according to claim 1, wherein at least one N-terminal end of the two amino acid chains forming the polypeptide is modified with a chemical group selected from the group consisting of one or two alkyl groups, such as methyl, ethyl, propyl or butyl groups, an acyl group, an acetyl or propionyl group, or the amino acid pyroglutamic acid forms the N-terminal end.

6. An auxiliary agent for the precipitation of viruses, containing a polypeptide according to claim 1.

7. A medicament comprising a polypeptide according to claim 1.

8. A method of gene therapy comprising administering to a patient a polypeptide of claim 1 to treat a genetically caused disease.

9. A process for enhancing the infection of a cell with a virus, comprising the steps of:
providing the polypeptide according to claim 1 dissolved in an organic solvent;
adding the polypeptide to an aqueous solution to form insoluble aggregates of the polypeptide;
mixing the solution from the preceding step; and
culturing cells in the presence of a polypeptide claim 1.

10. A method of enhancing infection of a cell with a virus comprising exposing a cell to a virus and the polypeptide of claim 1 to enhance infection of the cell with the virus.

11. A composition containing a polypeptide according to claim 1.


SEQUENCE LISTING (25 sequences)

SEQ ID NO: 1 - protein, 12 aa, human
QCKIKQIINMWQ

SEQ ID NO: 2 - protein, 4 aa, human
SNNI

SEQ ID NO: 3 - protein, 5 aa, human
SNNIT

SEQ ID NO: 4 - protein, 5 aa, human
NNITL

SEQ ID NO: 5 - protein, 6 aa, human
SNNITL

SEQ ID NO: 6 - protein, 4 aa, human
EVGK

SEQ ID NO: 7 - protein, 5 aa, human
EVGKA

SEQ ID NO: 8 - protein, 6 aa, human
EVGKAM

SEQ ID NO: 9 - protein, 7 aa, human
EVGKAMY

SEQ ID NO: 10 - protein, 8 aa, human
EVGKAMYA

SEQ ID NO: 11 - protein, 9 aa, human
EVGKAMYAP

SEQ ID NO: 12 - protein, 10 aa, human
EVGKAMYAPP

SEQ ID NO: 13 - protein, 11 aa, human
EVGKAMYAPPI

SEQ ID NO: 14 - protein, 12 aa, human
EVGKAMYAPPIE

SEQ ID NO: 15 - protein, 13 aa, human
EVGKAMYAPPIEG

SEQ ID NO: 16 - protein, 4 aa, human
PIEG

SEQ ID NO: 17 - protein, 5 aa, human
PPIEG

SEQ ID NO: 18 - protein, 6 aa, human
APPIEG

SEQ ID NO: 19 - protein, 7 aa, human
YAPPIEG

SEQ ID NO: 20 - protein, 8 aa, human
MYAPPIEG

SEQ ID NO: 21 - protein, 9 aa, human
AMYAPPIEG

SEQ ID NO: 22 - protein, 10 aa, human
KAMYAPPIEG

SEQ ID NO: 23 - protein, 11 aa, human
GKAMYAPPIEG

SEQ ID NO: 24 - protein, 12 aa, human
VGKAMYAPPIEG

SEQ ID NO: 25 - protein, 13 aa, human
EVGKAMYAPPIEG